(19) United States
(12) Patent Application Publication
(10) Pub. No.: US 2010/0063327 A1
(43) Pub. Date: Mar. 11, 2010

(54) PROCESS FOR REDUCING SIDE-REACTIONS DURING ALKYLENE GLYCOL AND POLY-ALKYLENE GLYCOL MANUFACTURING

(75) Inventors: Richard C. Hoy, Charleston, WV (US); Louis A. Kapicak, Cross Lanes, WV (US)

Correspondence Address: The Dow Chemical Company, Intellectual Property Section, P.O. Box 1967, Midland, MI, 48641-1967, US

(73) Assignee: DOW TECHNOLOGY INVESTMENTS LLC, Midland, MI (US)

(21) Appl. No.: 12/516,366

(22) PCT Filed: Dec. 12, 2007
(86) PCT No.: PCT/US2007/025492

§ 371 Date: Nov. 16, 2009

Related U.S. Application Data
(60) Provisional application No. 60/876,952, filed on Dec. 22, 2006.

(51) Int. Cl. C07C 41/02; C07C 41/01

(52) U.S. Cl. 568/613; 568/671

(57) ABSTRACT

This invention relates to a process of producing one or more of an alkylene glycol or poly-alkylene glycol by the reaction of an alkylene oxide and water whereby reduced levels of undesired by products such as carbonyl compounds, ultraviolet light absorbing compounds and various metal species are produced.

### FIELD OF THE INVENTION

The present invention relates to methods for producing an alkylene glycol by reaction of an alkylene oxide and water. It further relates to methods for producing higher glycols by reaction of an alkylene oxide and an alkylene glycol. The invention more specifically relates to methods for reducing the amounts of certain types of impurities and/or by-products that can be generated during the manufacture and subsequent purification of such glycols.

### BACKGROUND OF THE INVENTION

1,2-alkylene glycols are manufactured by heating a mixture of the corresponding alkylene oxide and water to an elevated temperature at which the water will react at the site of the epoxide group to form vicinal hydroxyl groups. This reaction may be effected with or without a catalyst. Thus, ethylene oxide and water react to form 1,2-ethylene glycol and propylene oxide and water react to form 1,2-propylene glycol. Common by-products of the reaction to produce 1,2-ethylene glycol include diethylene glycol (“DEG”) and triethylene glycol (“TEG”). Other, higher glycols can be are produced as well by, for example, by the reaction of DEG with an alkylene oxide. Tetraethylene glycol (“TETRA”) is an example of a higher glycol.

Various unwanted side reactions can occur during these processes. Carbonyl compounds often form via several mechanisms. For example, the alkylene glycol can oxidize to form the corresponding aldehyde plus a molecule of water. Additionally, for example, the alkylene glycol can dehydrogenate to form the corresponding aldehyde plus a molecule of hydrogen. These and other carbonyl compounds can subsequently react to form ultraviolet light absorbing compounds which must also be removed from the product.

Other unwanted side reactions can include the leaching of metal species from the process equipment as well as the formation of metal salts and metal oxides. In sufficient quantity, these metal species may be problematic in themselves, but even at low levels they may catalyze the oxidation and/or dehydrogenation of alkylene glycols or poly-alkylene glycols to form carbonyl compounds. Various metal species can form at virtually any stage in such glycol manufacturing processes and can be exacerbated by the presence of oxygen and/or acidic materials.

Carbonyl containing compounds often form downstream of the glycol reactor in finishing columns where the alkylene glycol (or dimers or higher oligomers such as DEG, TEG, and TETRA) is distilled. Here the problem may be related to the presence of low levels of oxygen that results in oxidation of such glycol to form carbonyl compounds. Additionally, the problem may be related to the presence of certain metal species which may act as catalysts. As mentioned above, these carbonyl compounds may further react to form ultraviolet light absorbing compounds. The problem of carbonyl compound formation during such glycol finishing is often more acute during start-up and shut-down operations, or when there are process upsets. The formation of carbonyl compounds and ultraviolet light absorbing compounds is a significant problem because of the need to remove both the carbonyl compounds and the ultraviolet light absorbing compounds from the glycol in order to satisfy the requirements of certain end use applications. This separation can be difficult and adds both capital and operating expense to the process.

One way of treating alkylene glycols to remove aldehydes is to contact the mixture with a bisulfite compound. For example, U.S. Pat. No. 6,187,973 describes a method for removing aldehydes from ethylene glycol by contacting the ethylene glycol with a bisulfite-treated anion exchange resin. Canadian Patent No. 1,330,350 describes adding bisulfite ions to an ethylene glycol mixture, followed by contacting the mixture with an anion exchange resin in the hydroxyl form, to remove aldehydes. JP 53-029292 describes a process for absorbing aldehydes from a gas stream, in which the stream is contacted with an activated carbon that is impregnated with a sulphite or acid sulfite salt. SU 1498752 (abstract) describes a method of purifying ethylene glycol with a first reagent mixture that contains sodium hypochlorite, bromine, p-chlorobenzenesulfonic acid dichloramine or N-chlorosuccinamide, and then treating the solution with a solution of sodium bisulfite. These processes all focus on removal methods rather than methods for reducing aldehyde (or other by-product) generation in the first instance. Research Disclosure 465117 (Kenneth Mason Publications, Ltd., January 2003) describes adding a reactant such as a sulphite to certain ethylene oxide/ethylene glycol process streams for impurity conversion. Bisulfite ions also have been added into processes for producing ethylene glycol from ethylene oxide, carbon dioxide and water via an ethylene carbonate intermediate.

What is needed is a process by which reduced levels of undesired by-products such as carbonyl compounds, ultraviolet light absorbing compounds and various metal species are produced in an alkylene glycol or poly-alkylene glycol production process.

### SUMMARY OF THE INVENTION

This invention relates to a process of producing one or more of an alkylene glycol or poly-alkylene glycol by the reaction of an alkylene oxide and water whereby reduced levels of undesired by products such as carbonyl compounds, ultraviolet light absorbing compounds and various metal species are produced. Specific process operations in which the process of the invention is particularly suitable include alkylene glycol reactors and alkylene glycol distillation units. Applicants have found that the presence of the water soluble reducing agent in many cases decreases the amounts of undesired side reactions that occur when the process stream is at the elevated temperature conditions. The formation of carbonyl compounds, such as aldehydes, metal species and ultraviolet light absorbing compounds is reduced when the reducing agent is present.

In one aspect, this invention is a method comprising subjecting a reaction mixture containing an alkylene oxide and water to reaction conditions including an elevated temperature sufficient to convert at least a portion of the alkylene oxide to one or more of the corresponding alkylene glycol or poly-alkylene glycol, wherein the reaction mixture further contains from 1 ppb to 5% by weight of the reaction mixture of a water soluble reducing agent. The method of this aspect tends to produce fewer carbonyl compounds, fewer ultraviolet light absorbing compounds and fewer metal species than when the reducing agent is not present. As a result, downstream purification processes are simplified and less expensive.

In another aspect, this invention is a method comprising the addition of the water soluble reducing agent to the process stream containing an alkylene oxide and one or more of water and poly-alkylene glycol after the process stream has been subjected to an elevated temperature sufficient to convert at least a portion of the alkylene oxide to one or more of the corresponding alkylene glycol or poly-alkylene glycol. The method of this aspect also tends to produce fewer carbonyl compounds, fewer ultraviolet light absorbing compounds and fewer metal species than when the reducing agent is not present and differs from the previous aspect in that reaction of the water soluble reducing agent with the alkylene oxide is significantly reduced. As a result, downstream purification processes are simplified and less expensive.

In still another aspect, this invention is a method comprising distilling a mixture containing an alkylene glycol or poly-alkylene glycol, wherein the mixture contains from 1 ppb to 5% by weight, of a water soluble reducing agent. In this aspect of the invention, the formation of aldehydes and of ultraviolet light absorbing compounds can be reduced quite significantly.

### DETAILED DESCRIPTION OF THE INVENTION

In this invention, a water soluble reducing agent is present in the reaction mixture at one or more stages of the process of manufacturing or distilling an alkylene glycol or poly-alkylene glycol.

The alkylene oxide is a 1,2-alkylene oxide such as ethylene oxide, propylene oxide, 1,2-butylene oxide, 1,2-hexene oxide and the like. The corresponding alkylene glycol is a vicinal dihydroxy alkane, such as 1,2-ethylene glycol, 1,2-propylene glycol, 1,2-butylene glycol, 1,2-hexane glycol and the like. The alkylene glycol of most interest is 1,2-ethylene glycol. The poly-alkylene glycol of most interest is diethylene glycol. The following discussion features alkylene glycols, but is also applicable to poly-alkylene glycols, particularly DEG, TEG, and TETRA.

During the manufacturing of such alkylene glycols and poly-alkylene glycols, process streams which contain the glycol are often subjected at one or more times to temperatures of 100° C. or above.

For example, temperatures exceeding 100° C. are often encountered in a reactor in which the alkylene glycol is formed from a precursor mixture of alkylene oxide and water. The alkylene glycol contained in the process stream may be primarily or even entirely that formed in that reactor. For example, process streams entering a reactor may contain precursor compounds (alkylene oxides and water, for example) but little or none of the glycol.

In the case of ethylene glycol manufacturing, for example, a mixture of ethylene oxide and water in the absence of a catalyst is usually subjected to a temperature of 100° C. or higher, under superatmospheric conditions sufficient to maintain the components of the stream (ethylene oxide, water and product ethylene glycol) in liquid form. Carbonyl compounds can form in the reactor under these conditions.

Another unit operation in which a glycol-containing process stream is subjected to such temperatures is a distillation unit, in which the alkylene glycol is distilled to separate it from impurities. An alkylene glycol production facility may contain more than one of these, and they are often arranged in series to conduct multiple distillations in order to produce a more purified product. In some glycol production facilities, crude glycol that exits a glycol reactor is sent through one or more evaporators, where much of the residual water is removed from the glycol. The process stream is then sent to one or more distillation columns where the water content is reduced to parts per million levels and other volatile impurities are removed. The temperatures in the distillation unit(s) generally range from 130° C. up to or exceeding the normal boiling temperature of the alkylene glycol. Ethylene glycol, for example, boils at about 197° C. and 1,2-propylene glycol boils at about 187° C., at 1 atmosphere pressure. Exposure of the alkylene glycol to these temperatures often leads to the development of impurities, particularly carbonyl compounds such as aldehydes, and ultraviolet light absorbing compounds.

There are at least three general classes of ultraviolet light absorbing compounds which are impurities: (1) the 1,2-cyclopentanediones, and in particular 3-methyl-1,2-cyclopentanedione; (2) the 1,3-cyclopentandediones, and in particular 4-methyl-1,3-cyclopentanedione; and (3) the cyclopentenones, and in particular 2-cyclopentenone.

Without limiting the invention to any theory, it is believed that formation of carbonyl compounds may be in some cases related to the presence of certain metal species such as, metal oxides, metal salts or metal ions that periodically can become present in certain reaction vessels. Metals that are capable of forming carbonyl compounds are of particular concern. Prominent examples of such metals are nickel and copper. It is believed that variations in the composition tend to occur most often at start up, shut down and during process upsets. It is believed that these metals, metal salts or oxides derived from these metals can be carried downstream into unit processes where high temperatures are encountered, at which point they catalyze the formation of carbonyl compounds. This particular problem is believed to account for a substantial amount of carbonyl compound formation in alkylene and poly-alkylene glycol distillation units.

Without limiting the invention to any theory, it is believed that through the use of this invention, the formation of these various types of byproducts is suppressed through the presence of the reducing agent in the process stream. In certain embodiments of the invention, reducing agent is advantageously present in the alkylene glycol or poly-alkylene glycol containing process stream at such time as the process stream is exposed to a temperature of 100° C. or higher. In other embodiments, the reducing agent is present in the process stream as it is subjected to alkylene glycol or poly-alkylene glycol forming conditions. In yet other embodiments, the reducing agent is present during a distillation of the alkylene glycol or poly-alkylene glycol.

The reducing agent is water soluble. It should not react significantly, under the conditions of the process, with any alkylene oxide or alkylene glycol or poly-alkylene glycol that is present, although some reaction can be tolerated if sufficient reducing agent is available to effect the desired result and if yield losses are not too high. Suitable reducing agents include, for example, water soluble sulfite, bisulfite, metabisulfite and phosphite compounds, as well as hydroxylamine. Water soluble sulfite, bisulfite and metabisulfite salts are preferred. Suitable alkali metal sulfite, bisulfite and metabisulfite salts include sodium sulfite, sodium bisulfite, sodium metabisulfite, potassium sulfite, potassium bisulfite, potassium metabisulfite cesium sulfite, cesium bisulfite, cesium metabisulfite, lithium sulfite, lithium bisulfite and lithium metabisulfite. The sodium and potassium sulfites, bisulfites and metabisulfites are preferred.

The reducing agent is generally benign to the overall process, and often can be introduced either into the process unit where it is needed, or at some upstream point from which it is carried through the process into the unit operations described before.

The reducing agent can be introduced upstream of the alkylene glycol or poly-alkylene glycol reactor or directly into such glycol reactor if it is desired to control the formation of impurities in the reactor. The reducing agent may also be introduced at the exit of such glycol reactor and/or downstream of the reactor (as in a downstream distillation unit). Alternatively the reducing agent can be introduced directly into an alkylene glycol or poly-alkylene glycol distillation unit, or at any upstream point from which it will carry through to the distillation unit, if control of impurity formation in the distillation unit is what is desired.

Under certain circumstances, the reducing agent may be generated in situ, by adding an appropriate precursor material. For example, sulfurous acid, sulfur dioxide, an organic ester of sulfurous acid, an addition product of a bisulfite or sulfite with an organic material, or an alkali metal salt thereof can be added to a process stream having a pH of greater than 7, to form sulfite or bisulfite ions in situ.

The amount of reducing agent can range widely. The reducing agent can constitute from as little as 1 part per billion or as much as 5%, based on the weight of the process stream being treated. Generally, excess amounts over what are needed are not harmful, although they can add unnecessary expense. The reducing agent can be added continuously or intermittently as needed to maintain effective levels.

It is often desirable to introduce the reducing agent only at such times that impurity formation is expected. These times include startups, shutdowns, or periods of process upset. Thus, for example, in some embodiments, the reducing agent may be added during the startup or shutdown phase of operation, or in response to process upsets, as a prophylactic measure to prevent potential impurity formation. In other embodiments of the invention, the presence of one or more impurities in the process streams is monitored. In such cases, the reducing agent can be added on an as-needed basis in response to the detection of the impurity or impurities. If desired, an effective level of the reducing agent can in these situations be maintained in the process streams during the entire period of operation as a prophylactic measure.

Preferred amounts may vary according to the particular point in the process where they are needed. The main matter of concern is often carbonyl compound formation. In such a case, a preferred amount is from 10 parts per billion to 5% by weight, a more preferred amount is from about 50 ppb to 3% by weight, and a most preferred amount especially from 100 ppb to 3% by weight.

It is not normally necessary to make further adjustments to the process of making the alkylene glycol or poly-alkylene glycol or distilling it, other than supplying an effective amount of the reducing agent to the appropriate process stream. Conditions for the alkylene glycol or poly-alkylene glycol forming reaction and subsequent processing of the product stream can be operated in the same manner as when the reducing agent is absent. Suitable conditions for reacting an alkylene oxide with water to form the corresponding alkylene glycol are described, for example, in U.S. Pat. Nos. 4,822,926, 3,922,314 and 6,514,388. Suitable conditions for operating an integrated ethylene oxide/ethylene glycol process are described, for example, in U.S. Pat. No. 6,437,199. The conditions described therein are generally suitable for use with this invention.

Typically, conditions of the reaction of the alkylene glycol and water to produce the alkylene oxide will include an elevated temperature, such as from 100 to 210° C., especially from 140 to 200° C. The reaction conditions also will typically include a superatmospheric pressure, such as from 200 psig to 500 psig (379 to 3448 kPa) or more. Water is usually present in stoichiometric excess, relative to the alkylene oxide. From 1 to 15 moles of water may be present per mole of alkylene oxide in the starting reaction mixture. The reaction may be catalyzed. Suitable catalysts for the reaction of alkylene oxides to, the corresponding glycols are described in U.S. Pat. No. 5,260,495.

The following examples are provided to illustrate the invention, but are not intended to limit the scope thereof. All parts and percentages are by weight unless otherwise indicated.

### EXAMPLE 1

To the inlet of an ethylene glycol reactor being fed 140 kg/s of ethylene oxide plus water at an approximate 1:14 weight ratio is fed 2.5 wt-% of a sodium sulfite/cobalt sulfate solution at a rate of 50 kg/h. Prior to addition of the sodium sulfite/cobalt sulfate solution, the distillate contains 14.5 ppm of carbonyl compounds calculated as acetaldehyde and has an ultraviolet light transmittance of 96.5% at 220 nm, 94.0% at 250 nm and 96.9% at 275 nm. After addition of the sodium sulfite/cobalt sulfate solution, the carbonyl content of the distillate drops to 10 ppm and the ultraviolet light transmittance increases to 98.1% at 220 nm, 96.8% at 250 nm and 98.6% at 275 nm.

### EXAMPLE 2

To the inlet of an ethylene glycol distillation column being fed 13.0 kg/s of crude ethylene glycol is fed 2.5 wt-% of a sodium sulfite/cobalt sulfate solution at a rate of 10 liter/h. Prior to addition of the sodium sulfite/cobalt sulfate solution, the distillate contains 14.0 ppm of carbonyl compounds calculated as acetaldehyde and has an ultraviolet light transmittance of 98.0% at 275 nm. After addition of the sodium sulfite/cobalt sulfate solution, the carbonyl content of the distillate drops to 1.5 ppm and the ultraviolet light transmittance increases to 99.5% at 275 nm.

1. A method of preventing or decreasing the formation of carbonyl compounds, ultraviolet light absorbing compounds and various metal species in a process stream, comprising heating the process stream containing one or more of an alkylene glycol or poly-alkylene glycol to a temperature of at least 100° C., wherein the process stream further contains from 1 part per billion (ppb) to 5% by weight of the process stream of a water soluble reducing agent.

2. The method of claim 1 wherein the alkylene glycol is ethylene glycol and the poly-alkylene glycol is diethylene glycol, triethylene glycol or tetraethylene glycol.

3. The method of claim 2 wherein the reducing agent is a water soluble sulfite, bisulfite, metabisulfite or phosphite compound, hydroxylamine or a mixture of two or more thereof.

4. The method of claim 3 wherein the reducing agent is sodium sulfite sodium bisulfite, sodium metabisulfite, potassium sulfite, potassium bisulfite, potassium metabisulfite, cesium sulfite, cesium bisulfite, cesium metabisulfite, lithium sulfite, lithium bisulfite, lithium metabisulfite or a mixture of two or more thereof.

5. The method of claim 4 wherein the process stream contains from 50 ppb to 3% by weight of the reducing agent.

6. A method of preventing or decreasing the formation of carbonyl compounds, ultraviolet light absorbing compounds and various metal species in a reaction mixture, comprising subjecting the reaction mixture containing an alkylene oxide and one or more of water or poly-alkylene glycol to reaction conditions including an elevated temperature sufficient to convert at least a portion of the alkylene oxide to the corresponding alkylene glycol, or to the corresponding poly-alkylene glycol wherein the reaction mixture further contains from 1 ppb to 5% by weight of the reaction mixture of a water soluble reducing agent.

7. The method of claim 6 wherein the alkylene oxide is ethylene oxide and the poly-alkylene glycol is diethylene glycol, triethylene glycol, or tetra ethylene glycol.

8. The method of claim 7 wherein the reducing agent is a water soluble sulfite, bisulfite, metabisulfite or phosphite compound, hydroxylamine or a mixture of two or more thereof.

9. The method of claim 8 wherein the reducing agent is sodium sulfite, sodium bisulfite, sodium metabisulfite, potassium sulfite, potassium bisulfite, potassium metabisulfite, cesium sulfite, cesium bisulfite, cesium metabisulfite, lithium sulfite, lithium bisulfite, lithium metabisulfite or a mixture of two or more thereof.

10. The method of claim 9 wherein the process stream contains from 50 ppb to 3% by weight of the reducing agent.

11. The method of claim 6 wherein the reducing agent is added to the process stream at a time that the process stream is subjected to reaction conditions including an elevated temperature sufficient to convert at least a portion of the alkylene oxide to one or more of the corresponding alkylene glycol or poly-alkylene glycol.

12. The method of claim 6 wherein the reducing agent is added to the process stream prior to subjecting the reaction mixture to reaction conditions including an elevated temperature sufficient to convert at least a portion of the alkylene oxide to one or more of the corresponding alkylene glycol or poly-alkylene glycol.

13. A method of preventing or decreasing the formation of carbonyl compounds, ultraviolet light absorbing compounds and various metal species in a mixture comprising distilling the mixture containing one or more of an alkylene glycol or poly-alkylene glycol, wherein the mixture contains from 1 ppb to 5% by weight, of an alkali metal bisulfite, alkali metal sulfite, alkali metal meta-bisulfite, or combination of two or more thereof.

14. The method of claim 13 wherein the alkylene glycol is ethylene glycol and the poly-alkylene glycol is polyethylene glycol.

15. The method of claim 14 wherein the reducing agent is a water-soluble sulfite, bisulfite, metabisulfite or phosphite compound, hydroxylamine, or a mixture of two or more thereof.

16. The method of claim 15 wherein the reducing agent is sodium sulfite, sodium bisulfite, sodium metabisulfite, potassium sulfite, potassium bisulfite, potassium metabisulfite, cesium sulfite, cesium bisulfite, cesium metabisulfite, lithium sulfite, lithium bisulfite, lithium metabisulfite or a mixture of two or more thereof.

17. The method of claim 16 wherein the process stream contains from 50 ppb to 3% by weight of the reducing agent.

18. The method of claim 1, further comprising monitoring the presence of one or more of carbonyl compounds, metal species and ultraviolet light absorbing compounds in the process stream, and adding the reducing agent in response to the detection of the carbonyl compounds, metal species, and ultraviolet light absorbing compounds.

19. The method of claim 6, further comprising monitoring the presence of carbonyl compounds, metal species, and ultraviolet light absorbing compounds in the process stream, and adding the reducing agent in response to the detection of the carbonyl compounds, metal species, and ultraviolet light absorbing compounds.

20. The method of claim 13, further comprising monitoring the presence of carbonyl compounds, metal species, and ultraviolet light absorbing compounds in the process stream, and adding the reducing agent in response to the detection of the carbonyl compounds, metal species, and ultraviolet light absorbing compounds.
